(19) United States
(12) Patent Application Publication
(10) Pub. No.: US 2024/0261458 A1
(43) Pub. Date: Aug. 8, 2024

(54) BANDAGE COMPONENT WITH SAP COATING

(71) Applicant: Biodaptive Advanced Materials, LLC, Ronkonkoma, NY (US)

(72) Inventors: Matthew Robert HENRY, State College, PA (US); Daniel RIVEST, Charlotte, VT (US); Patrick John LAMB, New Port Richey, FL (US); La YANG, Dunedin, FL (US)

(21) Appl. No.: 18/399,155

(22) Filed: Dec. 28, 2023

Related U.S. Application Data
(60) Provisional application No. 63/441,490, filed on Jan. 27, 2023.
(60) Provisional application No. 63/542,005, filed on Oct. 2, 2023.
(60) Provisional application No. 63/545,287, filed on Oct. 23, 2023.

(51) Int. Cl. A61L 15/42; A61F 13/0206; A61L 15/24; A61L 15/26; A61L 15/58; A61L 15/60

(52) U.S. Cl. CPC A61L 15/425; A61F 13/0209; A61L 15/24; A61L 15/26; A61L 15/58; A61L 15/60

(57) ABSTRACT

A medical bandage or dressing, including an absorbent layer with at least one layer of polyurethane foam including a superabsorbent polymer additive. The medical bandage or dressing may include one or more hydrophilic non-woven fabric layers bound to the polyurethane foam and one or more layers of the dressing may be heated and sealed to reduce shedding of the superabsorbent polymer additive as liquid is absorbed by the dressing.

### CROSS-REFERENCE TO RELATED APPLICATIONS

The present application claims benefit of and priority to U.S. Provisional Patent Application Ser. No. 63/441,490 filed Jan. 27, 2023 entitled BANDAGE WITH SUPER ABSORBENT POLYMER, U.S. Provisional Patent Application Ser. No. 63/542,005 filed Oct. 2, 2023 entitled BANDAGE COMPONENT WITH SAP COATING, and U.S. Provisional Patent Application Ser. No. 63/545,287 filed Oct. 23, 2023 entitled BANDAGE COMPONENT WITH SAP COATING, the entire content of each of which is incorporated by reference herein.

### BACKGROUND

### Field of the Disclosure

The present invention relates to a bandage or dressing for medical use that includes high fluid absorption. More particularly, the present invention relates to a bandage or dressing that includes an absorbent layer including one or more foam layers with a superabsorbent polymer (SAP) to absorb and retain exudate from the wound.

### Related Art

Bandages and dressings including absorbent material are typically provided to cover wounds, burns, bedsores and ulcers while they heal. Bandages and dressings may also be used as a preventative measure to prevent formation of sores or wounds. Conventional bandages typically include openings in an adhesive layer that contacts the user's skin to allow exudate from the wound to be drawn away from the wound site and absorbed in an absorbent layer that absorbs and retains the exudate. In conventional bandages and dressings, as the absorbent layer absorbs fluid, it tends to swell and stiffen which reduces flexibility of the bandage and tends to reduce patient comfort. Reduced flexibility not only affects comfort but may also reduce adhesions to the user's body as they move. Further, many conventional bandages have limited absorption capability such that frequent replacement may be necessary which is labor and time intensive and may increase the risk of infection or complications in the healing process.

Modern foam silicone dressings have revolutionized the industry in that they allow for the easy application of absorbent dressings and minimize trauma and scarring by providing an ideal moist environment for healing and comfort. However, the polyurethane foam may limit the amount of moisture that may be absorbed.

Accordingly, it would be beneficial to provide a bandage or dressing that is soft and comfortable while providing improved absorption and retention of wound exudate.

### SUMMARY

It is an object of the present disclosure to provide a foam dressing with improved comfort, absorbency and retention.

In embodiments, a medical dressing in accordance with an embodiment of the present disclosure includes: a carrier layer; an adhesive layer provided below the carrier layer closer to a user's skin when the medical dressing is in place on the user; an absorbent layer positioned below the adhesive layer and connected to the carrier layer via the adhesive layer, the absorbent layer configured to absorb and retain exudate and including at least one foam layer with superabsorbent polymer additive provided on the at least one foam layer; and a wound contact layer positioned below the absorbent layer and configured to adhere to the user's skin, the wound contact layer including a plurality of openings through which the exudate may pass into the absorbent layer.

In embodiments, the absorbent layer includes: a first polyurethane foam layer including at least a first layer of superabsorbent polymer additive; a second polyurethane foam layer including at least a second layer of superabsorbent polymer additive, wherein the exudate is absorbed by at least one of the first layer of superabsorbent polymer additive and the second layer of superabsorbent polymer additive.

In embodiments, at least one of the first layer of superabsorbent polymer additive and the second layer of superabsorbent polymer additive includes a binder powder configured to hold the superabsorbent polymer additive on the first polyurethane foam layer or the second polyurethane foam layer, respectively.

In embodiments, the first layer of superabsorbent polymer additive and the second layer of superabsorbent polymer additive are provided in power form and are impregnated into the first polyurethane foam layer and the second polyurethane foam layer, respectively.

In embodiments, at least one of the first layer of superabsorbent polymer additive includes superabsorbent polymer additive provided via an aqueous solution.

In embodiments, the first layer of superabsorbent polymer additive and the second layer of superabsorbent polymer additive include superabsorbent polymer additive provided via an aqueous solution.

In embodiments, the first layer of superabsorbent polymer additive and the second layer of superabsorbent polymer additive are provided via an aqueous solution applied to the first polyurethane foam layer and the second polyurethane foam layer, respectively.

In embodiments, the absorbent layer further comprises a third layer of superabsorbent polymer additive provided on at least one of the first polyurethane foam layer and the second polyurethane absorbent layer.

In embodiments, the third layer of superabsorbent polymer additive is a powder.

In embodiments, the third layer of superabsorbent polymer additive is provided via aqueous solution.

In embodiments, respective edges of the first polyurethane foam layer and the second polyurethane foam layer are sealed together to create a chamber to hold the superabsorbent polymer additive in place.

In embodiments, the respective edges are sealed via ultrasonic welding.

In embodiments, the dressing includes a hydrophilic non-woven fabric bound to at least one of the first polyurethane foam layer and the second polyurethane foam layer over the superabsorbent polymer additive.

In embodiments, the superabsorbent polymer additive is a powder impregnated in the at least one foam layer.

In embodiments, the superabsorbent polymer additive is provided via an aqueous solution.

In embodiments the superabsorbent polymer additive remains on the at least one polyurethane foam layer after a liquid of the aqueous solution evaporates.

In embodiments, the carrier layer is a polyurethane film.

In embodiments, the plurality of openings formed in the wound contact layer are all the same size.

In embodiments, the plurality of openings formed in the wound contact layer include a first group of openings having a large size positioned in a center of the dressing above the wound when the dressing is on the user's skin and a second group of openings having a small size positioned around a periphery of the dressing.

In embodiments, a first area of the carrier layer and a second area of the wound contact layer are larger than an area of the absorbent layer.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 a schematic diagram of a top view of a dressing in accordance with embodiments, of the present disclosure;

FIG. 2 is a schematic diagram of a top view of a dressing in accordance with other embodiments of the present disclosure;

FIG. 3 is a cross-sectional view of a dressing of FIG. 2 in accordance with embodiments of the present disclosure;

FIG. 4 is a cross-sectional view of an absorbent layer of a dressing in accordance with an embodiment of the present disclosure;

FIG. 5 is a cross-sectional view of an absorbent layer of a dressing in accordance with another embodiment of the present disclosure;

FIG. 6 is a cross-sectional view of an absorbent layer of a dressing in accordance with another embodiment of the present disclosure;

FIG. 6A is a cross-sectional view of an absorbent layer of a dressing in accordance with another embodiment of the present disclosure;

FIG. 6B is a cross-sectional view of an absorbent layer of a dressing in accordance with another embodiment of the present disclosure;

FIG. 7 is a cross-sectional view of an absorbent layer of a dressing in accordance with another embodiment of the present disclosure;

FIG. 7A is a cross-sectional view of an absorbent layer of a dressing in accordance with another embodiment of the present disclosure;

FIG. 8 is a cross-sectional view of an absorbent layer of a dressing in accordance with another embodiment of the present disclosure;

FIG. 8A is a cross-sectional view of an absorbent layer of a dressing in accordance with another embodiment of the present disclosure;

FIG. 9 is a cross-sectional view of an absorbent layer of a dressing in accordance with another embodiment of the present disclosure;

FIG. 9A is a cross-sectional view of an absorbent layer of a dressing in accordance with another embodiment of the present disclosure;

FIG. 10 is a cross-sectional view of an absorbent layer of a dressing in accordance with another embodiment of the present disclosure; and

FIG. 11 is a cross-sectional view of an absorbent layer of a dressing in accordance with another embodiment of the present disclosure.

### DETAILED DESCRIPTION OF THE EXEMPLARY EMBODIMENTS

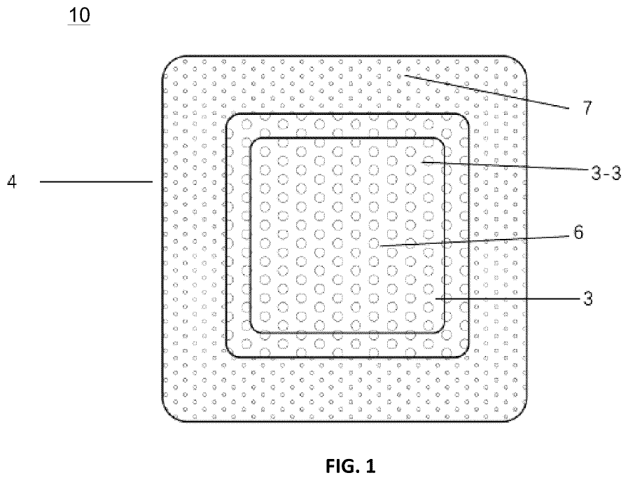

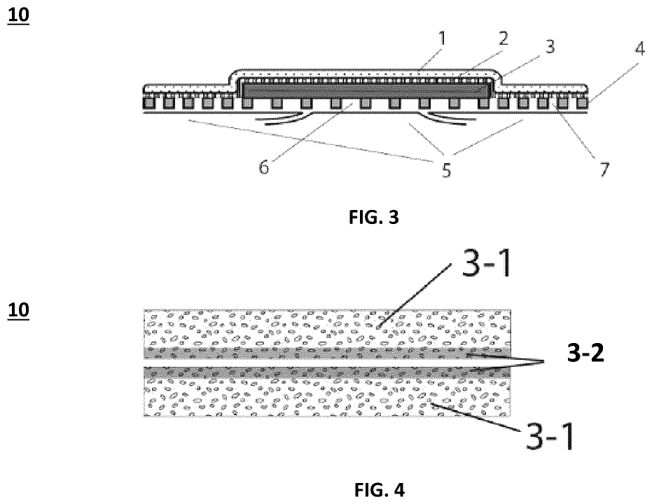

A wound care bandage or dressing 10 in accordance with an embodiment of the present application is illustrated in FIGS. 1-3, for example. In embodiments, the dressing 10 includes a carrier layer 1 (see FIG. 3, for example), which is attached to an adhesive layer 2. In embodiments, the carrier layer 1 may be a top film. In embodiments, the carrier layer 1 may be opaque or may be partially or fully transparent or translucent. In embodiments, the carrier layer 1 may be made of polyurethane. In embodiments, the carrier layer 1 may allow for evaporation of fluid from the bandage 10.

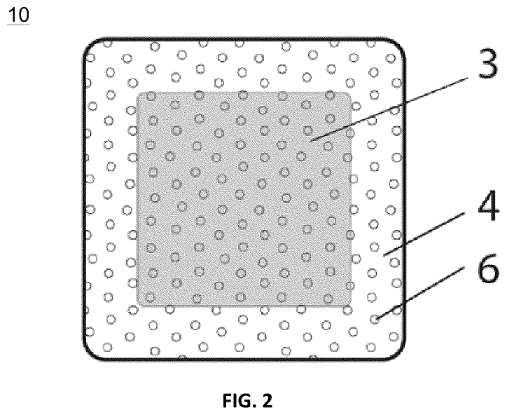

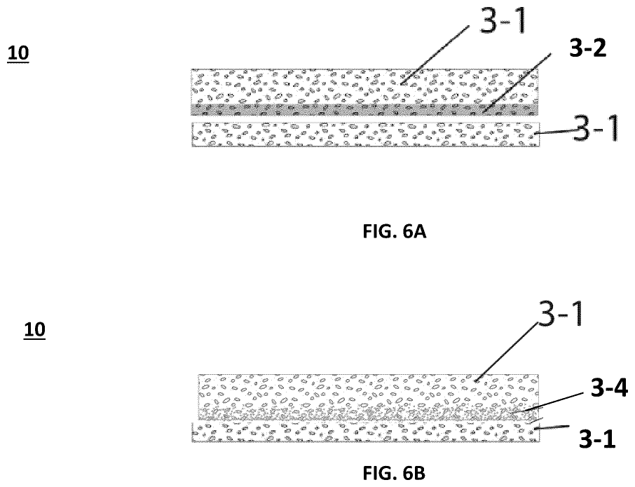

In embodiments, an absorption/retention layer 3 may be secured to the carrier layer 1 via the adhesive layer 2 as indicated in FIG. 3. In embodiments, the absorption/retention layer 3 may be referred to as simply the absorption layer. In embodiments, the absorption/retention layer 3 may absorb liquid exudate from a wound that is covered by the dressing 10. In embodiments, the absorption/retention layer 3 may be provided above a wound contact layer 4, which is suitable for contact with the user's skin when the dressing is in place on a user. In embodiments, the wound contact layer 4 may be a perforated silicone gel layer that may include large perforations 6 in a central portion which is generally positioned over the wound to allow fluid to pass from the wound and into the absorption/retention layer 3 as indicated in FIG. 2, for example. In embodiments, the dressing 10 may include smaller perforations 7 around the periphery of the dressing 10 to improve adhesion to the user's skin around the wound as indicated in FIGS. 1 and 3. For example. In embodiments, the dressing 10 may use the perforations 6 without the perforations 7 as indicated in FIG. 2. In embodiments, a removable protective layer 5 (see FIG. 3, for example) may be provided on the wound contact layer 4 prior to application of the dressing to the user. In embodiments, the protective layer 5 may be removed from the wound contact layer 4 to allow the dressing to be applied to the user's skin. While FIG. 4 illustrates the bandage or dressing 10 with both large openings 6 and small openings 7, the cross-sectional view of the dressing 10 using just the openings 6, as shown in FIG. 6 is the same as that as in FIG. 3 except that the openings 6 extend to the periphery of the wound contact layer 4.

In embodiments, the wound contact layer 4 may contain hydrogel or silicone gel, and the liquid absorption/retention layer 3 may include foam or highly absorbent fibers. The liquid absorption/retention layer 3, in embodiments, may also include a retention layer meant to retain exudate and prevent it from migrating back into the wound site.

### Embodiment 1

In embodiments, as shown in FIGS. 1, 2, 3 and 4, for example, the dressing 10 includes the carrier layer 1, the adhesive layer 2, the liquid absorption/retention layer 3, the wound contact layer 4 and the protective layer 5. In embodiments, the liquid absorption/retention layer 3 is arranged between the adhesive layer 2 and the wound contact layer 4 and the wound contact layer 4 may be a porous silicone gel layer. In embodiments, the protective layer 5 may be removable as noted above.

In embodiments, the area of the wound contact layer 4 and the carrier layer 1 is larger than that of the absorption/retention layer 3 which allows for a larger fitting area of the dressing around the wound to make application easier.

In embodiments, the protective layer 5 may be made of a polyethylene material, the carrier layer 1 may be made of a polyurethane material, and the adhesive layer 2 may be made of an acrylic adhesive.

In embodiments, the wound contact layer 4 may be a silicone gel layer including perforations 6 which may all be of the same size. In this embodiment, the wound contact layer 4 does not include the smaller openings 7, however, in embodiments, the smaller openings 7 may be used.

In embodiments, as can be seen in FIG. 4, for example, the absorption/retention layer 3 may include two layers of polyurethane foam 3-1, on which superabsorbent polymer additive powder 3-2 may be bonded by an adhesive/binder. In embodiments, the adhesive/binder increases the binding of polyurethane foam and superabsorbent polymer additive powder 3-2, minimizing shedding of superabsorbent polymer additive from the liquid absorption/retention 3 layer after absorbing liquid as well as during manufacture and use. In embodiments, a first layer of superabsorbent polymer additive powder 3.2 may be provided on a lower surface of the upper layer of foam 3-1 and a second layer may be provided on a top surface of the lower foam layer. In embodiments, the polyurethan foam layers 3-1 may be made of medical grade polyurethane foam, however, other foam materials may be used. In embodiments, the top surface of the upper first foam layer 3-1 may be connected to or otherwise positioned adjacent to the carrier layer 1, and the lower polyurethane foam layer 3-1 may be positioned below the upper layer closer to the user's skin when the bandage 10 is in place on the user's body. The lower polyurethane foam layer 3-1 may be connected to the wound contact layer 4 and aligned such that exudate from a wound under the dressing 10 passes through the openings 6 and into the absorbent layer 3. The superabsorbent polymer additive enhances absorption and retention of the exudate.

### Embodiment 2

In embodiments, as illustrated in FIGS. 1-3 and 5, for example, the dressing 10 may include the carrier layer 1, the adhesive layer 2, the liquid absorption/retention layer 3, the wound contact layer 4 and the protective layer 5. In embodiments, the liquid absorption/retention layer 3 may be provided between the adhesive layer 2 and the wound contact layer 4 and the wound contact layer 4 is a porous silicone gel layer. The protective layer 5 may be removable.

In embodiments, the area of the wound contact layer 4 and the carrier layer 1 is greater than the area of the liquid absorption/retention layer 3 to increase the fitting area of the dressing 10, as noted above.

In embodiments, the protective layer 5 may be made of a polyethylene material, the carrier layer 1 may be made of a polyurethane material, and the adhesive layer 2 may be made of an acrylic adhesive. In embodiments, the wound contact layer 4 may be silicone gel in which large perforation holes 6 and small perforation holes 7 are provided.

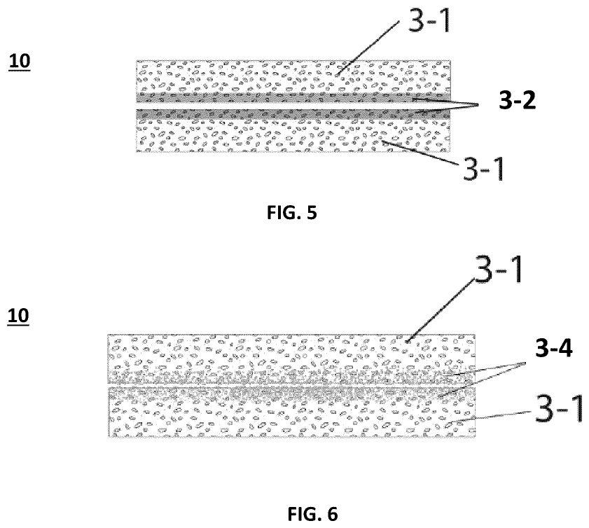

As can be seen in FIG. 5, for example, the liquid absorption/retention layer 3 may include two layers of polyurethane foam 3-1 with a surface of both polyurethane foam layers coated with a superabsorbent polymer additive layer 3-2 bonded by an adhesive/binder to minimize shedding of the SAP additive after absorbing liquid. In embodiments, four edges of the two layers of foam 3-1 may be heated and joined together using ultrasonic welding, for example, to form a chamber between the layers of foam 3-1 to prevent superabsorbent polymer additive from leaking from the liquid absorption/retention layer 3 after absorbing liquid or during manufacture or application. In embodiments, the foam layers 3-1 may be made of a similar material and positioned in a manner similar to that discussed above with respect to FIG. 4.

### Embodiment 3

In embodiments, as shown in FIGS. 1-3 and 6, for example, a high liquid absorption wound care dressing 10 may include the carrier layer 1, the adhesive layer 2 the liquid absorption/retention layer 3, the wound contact layer 4 and the protective layer 5. As noted above, in embodiments, the liquid absorption/retention layer 3 is arranged between the adhesive layer 2 and the wound contact layer 4 and the wound contact layer 4 is a porous silicone gel layer with the protective layer 5 removably connected thereto.

In embodiments, the area of the wound contact layer 4 and the carrier layer 1 is greater than the area of the liquid absorption layer 3 as noted above. In embodiments, the protective layer 5 is made of silicone coated release paper material, the carrier layer 1 is made of non-woven material, and the bonding layer 2 is made of acrylic adhesive.

In embodiments, the wound contact layer 4 may be made of an organic silicone rubber material, and the silicone gel layer may be provided with perforations 6 of the same size, as generally noted above.

As can be seen in FIG. 6, for example, the liquid absorption/retention layer 3 may be made of two layers of polyurethane foam 3-1 similar to that discussed above with respect to FIGS. 4 and 5. In embodiments, the surfaces of the polyurethane foam layers 3.1 that face each other may be coated with a superabsorbent polymer additive layer 3-4 applied in an aqueous solution. The superabsorbent polymer additive layers 3-4 may include an adhesive/binder to minimize shedding of superabsorbent polymer additive as liquid is absorbed.

In embodiments, the four edges of the two layers of foam 3-1 may be heated and bonded together using ultrasonic welding to form a chamber between the two layers of foam and further minimize shedding of superabsorbent polymer additive in a manner similar to that discussed above with respect to FIG. 5.

### Example 4

In embodiments, as shown in FIGS. 1-3 and 6A, for example, a liquid absorption wound care dressing 10 includes the carrier layer 1, the adhesive layer 2, the liquid absorption/retention layer 3, the wound contact layer 4 and the protective layer 5. In embodiments, the liquid absorption layer 3 is arranged between the adhesive layer 2 and the wound contact layer 4 and the wound contact layer 4 is a porous silicone gel layer. The protective layer 5 may be removable as noted above.

In embodiments, the area of the wound contact layer 4 and the carrier layer 1 is greater than the area of the liquid absorption layer 3. In embodiments, the protective layer 5 may be made of silicone coated release paper material, the carrier layer 1 may be made of non-woven material, and the bonding layer 2 may be or include acrylic adhesive.

In embodiments, the wound contact layer 4 may be made of an organic silicone rubber material, and the silicone gel layer is provided with perforations 6 of the same size.

In embodiments, as can be seen in FIG. 6A, for example, the liquid absorption/retention layer 3 may be made of two layers of polyurethane foam 3-1. In embodiments, the surface of one polyurethane foam layer 3-1 that faces the other polyurethane foam layer 3-1, the lower surface of the upper layer 3-1 in FIG. 6A, may be coated with a superabsorbent polymer additive powder layer 3-2. In embodiments, the lower foam layer 3-1 may not include a superabsorbent polymer powder layer 3-2 or 3-4. In embodiments, the lower foam layer 3-1 may include the superabsorbent polymer additive layer 3-2 and the upper foam layer may not include any superabsorbent polymer additive.

In embodiments, the superabsorbent polymer additive powder may be applied to the foam 3-1 via an aqueous/solvent-based coating 3-4 as can be seen in FIG. 6B, for example. Otherwise, the dressing 10 of FIG. 6B is the same as that of FIG. 6A with no superabsorbent polymer provided on the lower foam layer 3-1.

### Embodiment 5

In embodiments, as shown in FIGS. 1-3 and 7, for example, a high liquid absorption wound care dressing 10 may include the carrier layer 1, the adhesive layer 2, the liquid absorption/retention layer 3, the wound contact layer 4 and the protective layer 5. In embodiments, the liquid absorption/retention layer 3 is arranged between the adhesive layer 2 and the wound contact layer 4, and, as noted above the wound contact layer 4 is a porous silicone gel layer which may be removably covered by the protective layer 5.

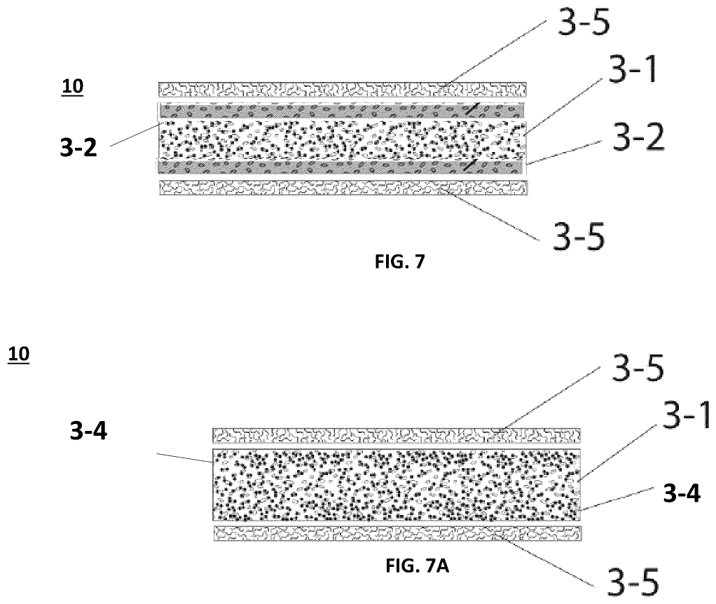

In embodiments, the area of the wound contact layer 4 and the carrier layer 1 is greater than the area of the liquid absorption layer 3. In embodiments, the protective layer 5 may be made of silicone coated release paper material, the carrier layer 1 may be made of non-woven material, and the adhesive layer 2 may be made of acrylic adhesive. In embodiments, as can be seen in FIG. 7, the wound contact layer 4 may be a silicone gel layer including perforation holes 6 and smaller perforation holes 7.

In embodiments, as can be seen in FIG. 7, for example, the liquid absorption/retention layer 3 may include one polyurethane foam layer 3-1 and hydrophilic non-woven fabric layers 3-5. In embodiments, the polyurethane foam layer 3-1 may be combined with superabsorbent polymer additive layer 3-2 provided as a powder. In embodiments, the polyurethane foam layer 3-1 and the non-woven layers 3-5 may be thermally bonded together to avoid the leaking of superabsorbent polymer additive from the liquid absorption/retention layer 3 after absorbing liquid. In embodiments, the superabsorbent polymer additive layer 3-2 may be provided as a powder impregnated into the foam.

In embodiments, the superabsorbent polymer additive may be provided as part of an aqueous/solvent-based coating 3-4 instead of as a powder, as can be seen in FIG. 7A, for example. The dressing 10 of FIG. 7A is otherwise the same as that illustrated in FIG. 7.

### Embodiment 6

In embodiments, as illustrated on FIGS. 1-3 and 8, a high liquid absorption wound care dressing 10 may include the carrier layer 1, the adhesive layer 2, the liquid absorption/retention layer 3, the wound contact layer 4 and the protective layer 5. In embodiments, the liquid absorption/retention layer 3 may be arranged between the adhesive layer 2 and the wound contact layer 4 and the wound contact layer 4 may be a porous silicone gel layer and the protective layer 5 may be removable.

In embodiments, the wound contact layer 4 and the carrier layer 1 may be greater than the area of the liquid absorption layer 3. In embodiments, the protective layer 5 may be made of silicone coated release paper material and the carrier layer 1 may be made of polyurethane film, and the adhesive layer 2 may be made of acrylic adhesive. In embodiments, the wound contact layer 4 may be a silicone gel layer in which perforation holes 6 of same size may be provided.

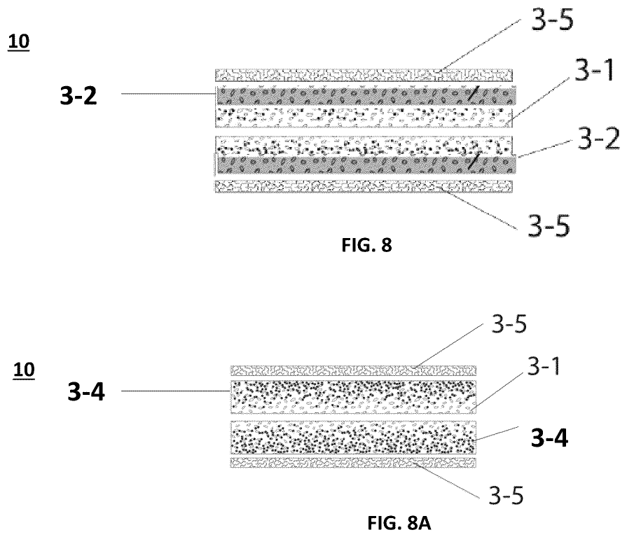

In embodiments, in FIG. 8, for example, the liquid absorption/retention layer 3 may be made of a two polyurethane foam layers 3-1 and hydrophilic non-woven fabric layers 3-5. In embodiments, the polyurethane foam layers 3-1 may be combined with superabsorbent polymer additive powder layer 3-2 provided on a top surface of the upper foam layer 3-1 and a lower surface of the lower foam layer 3-1. In embodiments, the polyurethane foam layers 3-1 and the non-woven layers 3-5 may be thermally bonded together to avoid leaking of superabsorbent polymer additive from the liquid absorption/retention layer 3 after absorbing liquid.

In embodiments, as shown in FIG. 8A, for example, the polyurethane foam layers 3-1 may be combined with a superabsorbent polymer additive layer 3-4 provided via an aqueous/liquid. Otherwise, the dressing 10 of FIG. 8A is the same as that described with reference to FIG. 8.

### Embodiment 7

In embodiments, as shown in FIGS. 1-3 and 9, a high liquid absorption wound care dressing 10 includes the carrier layer 1, the adhesive layer 2, the liquid absorption/retention layer 3, the wound contact layer 4 and the protective layer 5. In embodiments, the liquid absorption/retention layer 3 may be arranged between the adhesive layer 2 and the wound contact layer 4 and the wound contact layer 4 is a porous silicone gel layer with the protective layer 5 removably connected thereto.

In embodiments, the area of the wound contact layer 4 and the carrier layer 1 is greater than the area of the liquid absorption/retention layer 3. In embodiments, the protective layer 5 may be made of silicone coated release paper material and the carrier layer 1 may be made of polyurethane film while the adhesive layer 2 may be made of acrylic adhesive. In embodiments, the wound contact layer 4 may be a silicone gel layer in which perforation holes 6 of same size are provided.

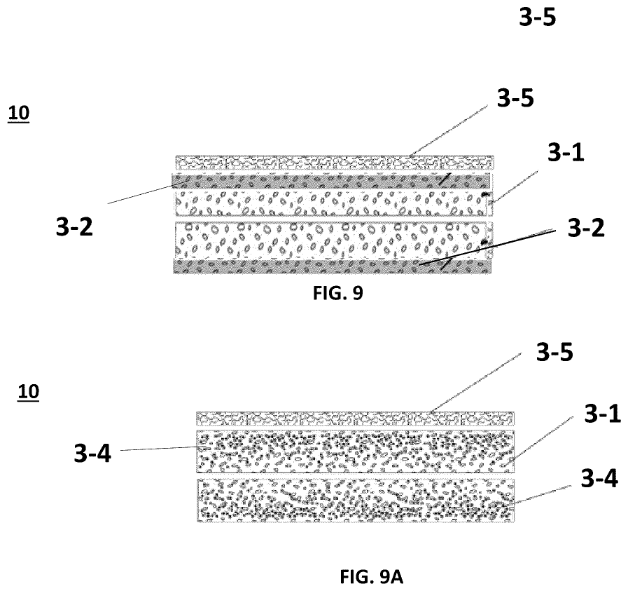

In embodiments, as can be seen in FIG. 9, the liquid absorption/retention layer 3 may be made of two layers of polyurethane foam 3-1 and one hydrophilic non-woven carrier fabric layer 3-5. In embodiments, hydrophilic non-woven fabric layer 3-5 may be bound to the surface of the polyurethane foam layer 3-1 that faces the carrier layer 1. In embodiments, the polyurethane foam layers 3-1 may be combined with superabsorbent polymer additive powder layer 3-2 which may be provided on the top surface of the upper foam layer 3-1 and a lower surface of the lower foam layer. In embodiments, the polyurethane foam layer 3-1 may be thermally bound to the non-woven layer 3-5 to avoid the leaking of superabsorbent polymer additive from the liquid absorption/retention layer after absorbing liquid.

In embodiments, as illustrated in FIG. 9A, the polyurethane foam layer 3-1 may be combined with superabsorbent polymer additive layer 3-4 provided via an aqueous liquid. Otherwise, the embodiment of FIG. 9A is the same as that described above with respect to FIG. 9.

### Embodiment 7

In embodiments, as shown in FIGS. 1-3 and 10, a high liquid absorption wound care dressing 10 may include the carrier layer 1, the adhesive layer 2, the liquid absorption/retention layer 3, the wound contact layer 4 and the protective layer 5. In embodiments, the liquid absorption/retention layer 3 may be arranged between the adhesive layer 2 and the wound contact layer 4 and the wound contact layer 4 may be a porous silicone gel layer. In embodiments, the protective layer 5 may be removable.

In embodiments, the area of the wound contact layer 4 and the carrier layer 1 is greater than the area of the liquid absorption/retention layer 3. In embodiments, the protective layer 5 may be made of silicone coated release paper material, the carrier layer 1 may be made of polyurethane film, and the bonding layer 2 may be made of acrylic adhesive. In embodiments, the wound contact layer 4 may be a silicone gel layer in which perforation holes 6 of same size are provided.

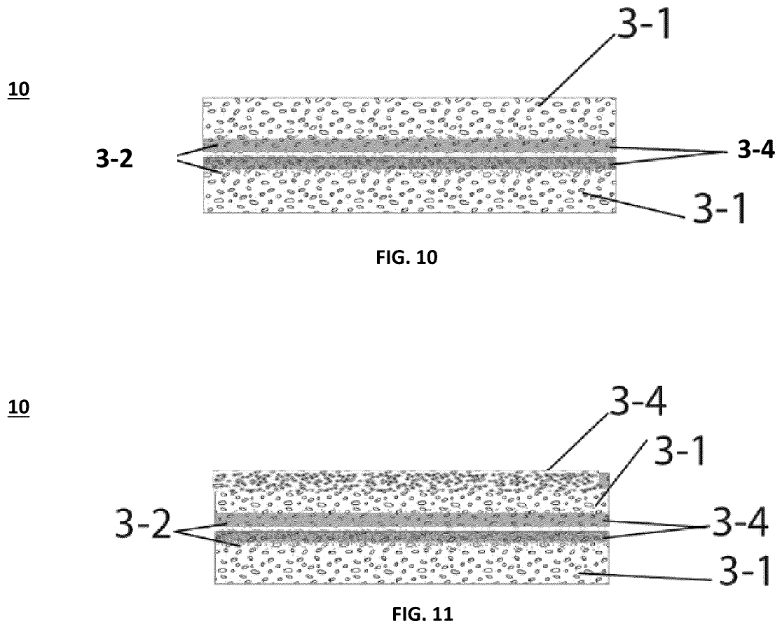

In embodiments, as can be seen in FIG. 10, for example, the liquid absorption/retention layer 3 may be made of two polyurethane foam layers 3-1. In embodiments, the surfaces of each polyurethane foam layer 3-1 facing each other may be coated with an aqueous/solvent based superabsorbent polymer additive layer 3-4. In embodiments, the aqueous/solvent on the foam layer 3-1 may be dried and a secondary surface treatment of layer 3-2 of superabsorbent polymer additive powder is applied to the foam using a powder-based treatment. In embodiments, the four edges of the two layers of polyurethane foam layers 3-1 may be joined by an ultrasonic seal to form a chamber between the two layers of foam 3-1 to avoid the leaking of superabsorbent additive from the liquid absorption/retention layer 3 after absorbing liquid.

### Embodiment 8

As can be seen in FIGS. 1-3 and 11, a high liquid absorption wound care dressing 10 includes the carrier layer 1, the adhesive layer 2, the liquid absorption/retention layer 3, the wound contact layer 4 and the protective layer 5. In embodiments, the liquid absorption/retention layer 3 may be arranged between the adhesive layer 2 and the wound contact layer 4 where the wound contact layer 4 may be a porous silicone gel layer and the protective layer 5 may be removable.

In embodiments, the wound contact layer 4 and the carrier layer 1 is greater than the area of the liquid absorption layer 3. In embodiments, the protective layer 5 may be made of silicone coated release paper material, the carrier layer 1 may be made of polyurethane film, and the bonding layer 2 may be made of acrylic adhesive. In embodiments, the wound contact layer 4 may be a silicone gel layer in which perforation holes 6 of same size are provided.

In embodiments, as can be seen in FIG. 11, the liquid absorption/retention layer 3 may include two polyurethane foam layers 3-1. In embodiments, both surfaces of the polyurethane foam layers 3-1 spaced from the wound contact layer 4 may be coated with an aqueous/solvent-based superabsorbent polymer additive layer 3-4. In embodiments, a bottom surface of the upper polyurethane foam layer 3-1 closer to the wound contact layer 4 and the top layer of the lower polyurethane foam layer 3-1 may be coated with an aqueous/solvent-based superabsorbent polymer additive layer 3-4. In embodiments, the two layers 3-1 of polyurethane foam may be combined with superabsorbent polymer additive powder layer 3-2 using a powder-based treatment approach provided on the surfaces that face each other. In embodiments, the four edges of the two layers of polyurethane foam 3-1 may be joined by ultrasonic welding to form a chamber between the two layers of polyurethane foam to avoid leaking of superabsorbent polymer additives from the liquid absorption/retention layer 3 after absorbing liquid.

In each of the embodiments discussed herein, exudate and moisture from the wound may be absorbed in the absorbent/retention layer 3. In particular, exudate and moisture will pass through the foam layer 3-1 and into the superabsorbent polymer additive layers 3-2, 3-4. As the superabsorbent polymer absorbs exudate, it may expand slightly and provide a gel like consistency. In embodiments, the gel like consistency allows the absorbent/retention layer 3 to remain flexible, and along with the use of foam layers 3-1, provides a cushion to improve user comfort. In embodiments, the use of superabsorbent polymer additive generally allows for absorption of substantial moisture, often more than other absorbent materials. In embodiments, absorbency may be measured following the BS EN 13726-1 method and absorbency may be in the range of 0.05 g/cm² to 3 g/cm², however, more or less absorbency may be provided.

In embodiments, as noted above, the wound contact layer 4 may be a porous silicone gel layer. In embodiments, the wound contact layer 4 may be implemented using a clear polyurethane film with a silicone based adhesive provided at least on a bottom surface thereof to secure the bandage or dressing 10 to the user's skin. In embodiments, the wound contact layer 4 may be adhered to the absorbent/retention layer 3, or more particularly, to the lower foam layer 3-1.

In embodiments, the silicone adhesive, or silicone gel of the wound contact layer 4 adheres to the user's skin around the wound while avoiding a strong bond with the wound. In embodiments, the wound contact layer 4 may include a plurality of perforations 6, 7 formed therethrough. In embodiments, the perforations 6, 7 allow exudate from the wound to pass through the wound contact layer 4 into the absorbent/retention layer 3, and specifically through the lower foam layer 3-1 and into the superabsorbent polymer additive layers 3-2, 3-4, where moisture is absorbed. In embodiments, the perforations 6, 7 may be provided in different shapes and sizes, for example, with smaller sized perforations 7 provided around a periphery of the bandage 10 to encourage good adhesion to the user's skin around the wound and large sized perforations 6 provided in the center, over the wound, to allow more exudate to be absorbed.

In embodiments, other materials may be used to make the wound contact layer 4, provided that the adhesive that faces the user's skin is silicone based. In embodiments, any adhesive that is suitable for contact with a user's skin may be used, including medical grade silicone adhesive, medical grade acrylic adhesive, and medical grade hydrocolloid adhesive, to name a few. In embodiments, the perforations 6 allow exudate to pass therethrough and into the absorbent/retention layer 3. The wound contact layer 4 provides a stable and firm connection to the user's skin to keep the bandage 10 in place while being relatively comfortable. In addition, the silicone adhesive allows for relatively easy removal, without damaging the wound.

In embodiments, a packaging element (not shown) may be provided to store the bandage or dressing 10 before use. In embodiments, the packaging element may be made of a combination of paper and polyethylene, or paper and forms a pouch or pocket in which the bandage 10 may be stored. In embodiments, the packaging element may be made of any other suitably durable material. In embodiments, the packaging element may be made of a material that is suitable to allow for sterilization of the bandage 10 while in place in the packaging element. In embodiment the packaging element may be made of a material that is impervious to liquids. In embodiments, the packaging element may be made of a material that allows liquid to exit, but not enter, or that allows liquid to enter but not exit. In embodiments, the packaging element may be transparent or include transparent portions. In embodiments, the packaging element may include an upper part and a lower part, with the bandage 10, including the layers discussed above, sandwiched between the upper and lower parts.

In embodiments, the use of the superabsorbent polymer additive between the lower foam layer and the upper foam layer 3-1, or elsewhere, provides for a gel cushion to form as exudate is absorbed. This gel consistency increases patient comfort and maximizes flexibility of the bandage, which allows for a better fit and reduces the likelihood of the bandage or dressing 10 separating from the user's skin. In embodiments, the use of the superabsorbent polymer additive layer 3-2, 3-4 provides for increased absorbency while at the same time generally reducing odor as the exudate is absorbed into the gel. In addition, the gel consistency of the superabsorbent polymer additive 3-2, 3-4 provides improved pressure distribution when force is applied to the bandage 10.

The bandage or dressing 10 may be prepared by impregnating the polyurethane foam layers 3-1 with the superabsorbent polymer additive powder and binding powder as noted above. In embodiments, samples were evaluated for the ability of the superabsorbent polymer additive powder and binding powder to remain bound to the substrate material after mild agitation that is meant to simulate manufacturing processes that occur post-impregnation, shipping/handling of that dressing, and/or use of the final finished bandage.

In embodiments an impregnation process used to impregnate the superabsorbent polymer additive powder and binding powder into the polyurethane foam layers 3-1 may be varied by adjusting parameters (e.g., magnitude of the AC electrical field) to drive the superabsorbent polymer additive and binding powder additive deeper into the foam matrix of the foam, thereby allowing more additive to be applied. In embodiments, both top and bottom surfaces of at least one foam layer 3-1 may be coated to increase the SAP/binder powder coating concentration. In embodiments, any suitable process may be used to impregnate the superabsorbent polymer additive powder and binding powder into the foam 3-1.

In embodiments, two wound exudate management properties may be examined: free-swell absorptive capacity (FAC) and fluid retention under compression (FRUC). FAC provides an assessment of the maximum absorptive capacity that can be achieved by a dressing material when exposed to an excess amount of simulated wound exudate in an in vitro environment. Whereas FRUC provides an in vitro evaluation of the quantity of fluid a wound dressing material can “lock away” while under a level of compression commonly used with venous leg ulcer bandages. Fluid retention is important because it tests a dressing material's ability to prevent leakage and fluid pooling, which could lead to negative outcomes in wound healing. Through the in vitro evaluation of both the absorptive capacity and fluid retention properties of each test article, assessment of how each article will likely manage wound exudate may be made.

A 2.5 mm EX polyurethane foam may be prepared to include various dry coat weights of superabsorbent polymer (SAP) powder and binding powder to provide different test articles. In embodiments, the SAP powder and the binding powder may be applied to the EX foam at 4 different ratios; (1) 80% SAP and 20% binder; (2) 90% SAP and 10% binder; (3) 95% SAP and 5% binder; and (4) 100% SAP and 0% binder. In embodiments, a non-treated 2.5 mm EX foam material may be prepared as a negative control article.

Table 1 provides additional information regarding the test and control articles:

| | Negative Control | Test Article 1 | Test Article 2 | Test Article 3 | Test Article 4 |
|---|---|---|---|---|---|
| Sample ID Number | N/A | 5IS1 | 5IS2 | 5IS5 | 5IS6 |
| Substrate Material | 2.5 mm EX foam | 2.5 mm EX foam | 2.5 mm EX foam | 2.5 mm EX foam | 2.5 mm EX foam |
| Functional Powder | None | 80% SAP | 90% SAP | 95% SAP | 100% SAP |
| | | (CA180N) | (CA180N) | (CA180N) | (CA180N) |
| Binding Powder | None | 20% PEG 8000PF | 10% PEG 8000PF | 5% PEG 8000PF | None |
| | | <100 μm | <100 μm | <100 μm | |
| Powder Amount | N/A | 474 gsm | 480 gsm | 441 gsm | 403 gsm |
| Estimated SAP | N/A | 379.2 gsm | 432.0 gsm | 419.0 gsm | 403.0 gsm |
| Powder Amount | | | | | |
| (Powder amount × SAP conc.) | | | | | |
| Shaken | No | Yes | Yes | No | No |
| Quantity Provided | N/A | 10.75 × 7.50 in | 10.75 × 7.50 in | 10.75 × 7.50 in | 10.75 × 7.50 in |
| | | (27.3 × 19.1 cm) | (27.3 × 19.1 cm) | (27.3 × 19.1 cm) | (27.3 × 19.1 cm) |

In embodiments, samples measuring 5 cm×5 cm may be cut from the central region of the polyurethan EX foam to provide each test article and control article. These articles were provided for both the free-swell absorptive capacity testing and subsequent fluid retention capacity testing. In embodiments, other sample sizes may be used.

In embodiments, in order to evaluate the ability of the powder additive(s) to remain attached/fixed to the polyurethane EX foam following the powder impregnation process and subsequent heat treatment, a shedding test may be performed. In embodiments, during the shedding test, the test article is attached to an arm of a “wrist action” shaker to provide a quick back and forth agitation motion to remove any non-bound or loosely bound powder additive and the test articles are shaken for 30 seconds. The weight of each test article both before and after the test may be recorded and compared to determine how much powder is lost. In embodiments, other shedding tests may be used.

In embodiments, to assess the capacity of different test articles to absorb fluid from moderately to heavily exuding wounds, a protocol based on BS EN 13726-1:2002, section 3.2[1] may be used. In short, wound exudate may be simulated by an aqueous test solution containing 142 mmol sodium and 2.5 mmol calcium as chloride salt (Test Solution A). This solution may have an ionic composition comparable to wound exudate. In embodiments, other simulated wound exudate may be used. A 5 cm×5 cm sized portion of the test article may be cut and measured to the nearest 0.1 cm to determine the exact sample area (A) and weighed in a dry state to determine a first mass (Mo). In embodiments, other sample sizes may be used. In embodiments, the test articles may be placed in a petri dish and immersed completely in a volume of test solution, prewarmed to 37° C.±1° C., that corresponded to 40 times the mass of the sample being examined. The test articles in the petri dishes may be transferred to an incubator and allowed to soak for 30 min 1 min at 37° C.±1° C. At the end of the incubation period, each sample is removed from the test solution using self-tensioning tweezers to grip the sample by one corner or by one end as appropriate and excess fluid allowed to drip off for 30 s±5 s seconds. The test articles were then reweighed to provide a second mass (Mₜ). In embodiments, absorptive capacity may be expressed as the mass of solution absorbed per 100 cm² and is calculated as follows:

$$Absorptive Capacity ( \frac{g}{100 {cm}^{2}} ) = \frac{( {M}_{t} - {M}_{0} ) \times 100}{A}$$

In embodiments, testing may be performed on a total of 3 replicate samples of each test article. In embodiments, the average absorptive capacity value for each test article, and relevant statistical data, may be recorded. In embodiments, other protocols or tests may be used.

In order to assess the ability of the test articles to retain fluid when under compression, a custom test may be performed based on the procedure proposed by Debashsish et al. and Foster et al. [2], [3], however, other procedures may be used. After carrying out a free swell test, each test article may be placed onto a polypropylene perforated surface, patient contacting surface (lower layer) facing downward, which was suspended over a container that could collect any fluid released by the test article sample during the test. A 7 cm×7 cm Plexiglass compression plate may be placed on top of the test article sample. The Plexiglass compression plate is used to help ensure homogenous pressure distribution over the entire sample. A pressure equivalent to the recommended compressive force used for venous leg ulcer compression bandaging (40 mm Hg) is applied to the test article sample using weights amounting to 1,350 g. After 1 min of compression, the setup consisting of the weights, plexiglass compression plate, test article, and PP perforated surface may be disassembled, and the test article sample may be reweighed to provide a third mass (Mr). Fluid retention capacity may be expressed as the mass of solution retained per 100 cm2 and calculated as follows:

$$Fluid Retention Capacity = ( \frac{g}{100 {cm}^{2}} ) = \frac{( {M}_{r} - {M}_{0} ) \times 100}{A}$$

Additionally, the percentage of fluid retention is calculated by comparing the ratio of the fluid held after compression (Mᵣ) to the absorptive capacity (Mₜ).

$$\% Fluid Retention = \frac{( {M}_{r} )}{( {M}_{t} )} \times 100 \%$$

In embodiments, testing is performed on a total of 3 replicate samples of each test article. The average absorptive capacity value for each test article, and relevant statistical data, may be reported as indicated in the below tables.

Test data from the fixation/shedding test conducted on the four test articles is summarized in Table 2 below:

TABLE 2
Summary of the fixation test data for the negative control article and test articles.

| | Powder | Powder | % Change in powder |
|---|---|---|---|
| | amount prior to | amount after | amount during |
| | shedding test | shedding test | shedding test |
| | (gsm) | (gsm) | (c) = [(a) − |
| Sample ID | (a) | (b) | (b)] / (a) |
| Negative Control 1 | N/A | N/A | N/A |
| Non-Treated EX 2.5 mm Foam | | | |
| (0% SAP / 0% Binder) | | | |
| Test Article 1 - 5IS1 | 484 | 474 | −2.07% |
| (80% SAP / 20% Binder) | | | |
| Test Article 2 - 5IS2 | 492 | 480 | −2.44% |
| (90% SAP / 10% Binder) | | | |
| Test Article 3 - 5IS3 (Similar to 5IS5) | 447 | 441 | −1.34% |
| (95% SAP / 5% Binder) | | | |
| Test Article 4 - 5IS4 (Similar to 5IS6) | 455 | 284 | −37.58% |
| (100% SAP / 0% Binder) | | | |

Test data from the free-swell absorptive capacity and fluid retention capacity tests conducted on the negative control and all four test articles are summarized in Tables 3, 4, 5, 6, and 7 provided below. Additionally, a summary of the average absorbency capacity, fluid retention, and percent change in average absorbency capacity relative to the negative control is provided in Table 8.

TABLE 3
Free-swell absorptive capacity and fluid retention capacity for the control article

| | | | Sample | Total | | | | |
|---|---|---|---|---|---|---|---|---|
| | | | Weight | Weight | Total | Free-swell | | |
| | | | (before | (after | Weight | absorptive | Fluid retention | |
| | | | absorbing | absorbing | Following | capacity | capacity | Percent fluid |
| | Sample | Area | liquid) | liquid) | Compression | (g/100 cm²) | (g/100 cm²) | retention |
| | Size | (cm²) | (g) | (g) | (g) | FAC = | FRC = | % FR = |
| Replicate No. | (cm) | (A) | (M₀) | (Mₜ) | (Mᵣ) | [(Mₜ − M₀) × 100]/A | [(Mᵣ − M₀) × 100]/A | (Mᵣ/Mₜ) × 100% |
| 1 | 5.0 × 5.0 | 25 | 0.3147 | 3.9635 | 3.8023 | 14.5952 | 13.9504 | 95.93% |
| 2 | 5.0 × 5.0 | 25 | 0.3312 | 4.4178 | 4.1513 | 16.3464 | 15.2804 | 93.97% |
| 3 | 5.0 × 5.0 | 25 | 0.3237 | 4.1956 | 3.7096 | 15.4876 | 13.5436 | 88.42% |
| Average Value | | | | | | 15.4764 | 14.2581 | 92.77% |
| Standard | | | | | | 0.8757 | 0.9084 | 3.90% |
| Deviation | | | | | | | | |
| Cofficient of | | | | | | 5.66% | 6.37% | 4.20% |
| Variation (CV) | | | | | | | | |

TABLE 4
Free-swell absorptive capacity and fluid retention capacity for the test article 1
(80% SAP, 20% binder)

| | | | Sample | Total | | | | |
|---|---|---|---|---|---|---|---|---|
| | | | Weight | Weight | Total | Free-swell | | |
| | | | (before | (after | Weight | absorptive | Fluid retention | |
| | | | absorbing | absorbing | Following | capacity | capacity | Percent fluid |
| | Sample | Area | liquid) | liquid) | Compression | (g/100 cm²) | (g/100 cm²) | retention |
| | Size | (cm²) | (g) | (g) | (g) | FAC = | FRC = | % FR = |
| Replicate No. | (cm) | (A) | (M₀) | (Mₜ) | (Mᵣ) | [(Mₜ − M₀) × 100]/A | [(Mᵣ − M₀) × 100]/A | (Mᵣ/Mₜ) × 100% |
| 1 | 5.0 × 5.0 | 25 | 1.5312 | 7.0176 | 6.8491 | 21.9456 | 21.2716 | 97.60% |
| 2 | 5.0 × 5.0 | 25 | 1.5338 | 7.422 | 7.2259 | 23.5528 | 22.7684 | 97.36% |
| 3 | 5.0 × 5.0 | 25 | 1.5584 | 7.2215 | 7.0258 | 22.6524 | 21.8696 | 97.29% |
| Average Value | | | | | | 22.7169 | 21.9699 | 97.42% |
| Standard | | | | | | 0.8055 | 0.7534 | 0.16% |
| Deviation | | | | | | | | |
| Cofficient of | | | | | | 3.55% | 3.43% | 0.17% |
| Variation (CV) | | | | | | | | |

TABLE 5
Free-swell absorptive capacity and fluid retention capacity for the test article 2
(90% SAP, 10% binder)

| | | | Sample | Total | | | | |
|---|---|---|---|---|---|---|---|---|
| | | | Weight | Weight | Total | Free-swell | | |
| | | | (before | absorbing | Weight | absorptive | Fluid retention | |
| | | | absorbing | (after | Following | capacity | capacity | Percent fluid |
| | Sample | Area | liquid) | liquid) | Compression | (g/100 cm²) | (g/100 cm²) | retention |
| | Size | (cm²) | (g) | (g) | (g) | FAC = | FRC = | % FR = |
| Replicate No. | (cm) | (A) | (M₀) | (Mₜ) | (Mᵣ) | [(Mₜ − M₀) × 100]/A | [(Mᵣ − M₀) × 100]/A | (Mᵣ/Mₜ) × 100% |
| 1 | 5.0 × 5.0 | 25 | 1.6063 | 7.6893 | 7.4217 | 24.332 | 23.2616 | 96.52% |
| 2 | 5.0 × 5.0 | 25 | 1.557 | 8.183 | 7.4577 | 26.504 | 23.6028 | 91.14% |
| 3 | 5.0 × 5.0 | 25 | 1.5691 | 8.0701 | 7.697 | 26.004 | 24.5116 | 95.38% |
| Average Value | | | | | | 25.6133 | 23.792 | 94.34% |
| Standard | | | | | | 1.1375 | 0.6461 | 2.84% |
| Deviation | | | | | | | | |
| Cofficient of | | | | | | 4.44% | 2.72% | 3.01% |
| Variation (CV) | | | | | | | | |

TABLE 6
Free-swell absorptive capacity and fluid retention capacity for the test article 3
(95% SAP, 5% binder)

| | | | Sample | Total | | | | |
|---|---|---|---|---|---|---|---|---|
| | | | Weight | Weight | Total | Free-swell | | |
| | | | (before | (after | Weight | absorptive | Fluid retention | |
| | | | absorbing | absorbing | Following | capacity | capacity | Percent fluid |
| | Sample | Area | liquid) | liquid) | Compression | (g/100 cm²) | (g/100 cm²) | retention |
| | Size | (cm²) | (g) | (g) | (g) | FAC = | FRC = | % FR = |
| Replicate No. | (cm) | (A) | (M₀) | (Mₜ) | (Mᵣ) | [(Mₜ − M₀) × 100]/A | [(Mᵣ − M₀) × 100]/A | (Mᵣ/Mₜ) × 100% |
| 1 | 5.0 × 5.0 | 25 | 1.4391 | 9.1251 | 8.5444 | 30.744 | 28.4212 | 93.64% |
| 2 | 5.0 × 5.0 | 25 | 1.3812 | 8.3126 | 7.8123 | 27.7256 | 25.7244 | 93.98% |
| 3 | 5.0 × 5.0 | 25 | 1.4218 | 8.6436 | 8.082 | 28.8872 | 26.6408 | 93.50% |
| Average Value | | | | | | 29.1189 | 26.9288 | 93.71% |
| Standard | | | | | | 1.5225 | 1.3713 | 0.25% |
| Deviation | | | | | | | | |
| Cofficient of | | | | | | 5.23% | 5.09% | 0.26% |
| Variation (CV) | | | | | | | | |

TABLE 7
Free-swell absorptive capacity and fluid retention capacity for the test article 4
(100% SAP, 0% binder)

| | | | Sample | Total | | | | |
|---|---|---|---|---|---|---|---|---|
| | | | Weight | Weight | Total | Free-swell | | |
| | | | (before | absorbing | Weight | absorptive | Fluid retention | |
| | | | absorbing | (after | Following | capacity | capacity | Percent fluid |
| | Sample | Area | liquid) | liquid) | Compression | (g/100 cm²) | (g/100 cm²) | retention |
| | Size | (cm²) | (g) | (g) | (g) | FAC = | FRC = | % FR = |
| Replicate No. | (cm) | (A) | (M₀) | (Mₜ) | (Mᵣ) | [(Mₜ − M₀) × 100]/A | [(Mᵣ − M₀) × 100]/A | (Mᵣ/Mₜ) × 100% |
| 1 | 5.0 × 5.0 | 25 | 1.1811 | 8.3991 | 8.0495 | 28.872 | 27.4736 | 95.84% |
| 2 | 5.0 × 5.0 | 25 | 1.1335 | 8.0742 | 7.592 | 27.7628 | 25.834 | 94.03% |
| 3 | 5.0 × 5.0 | 25 | 1.1974 | 8.4494 | 7.9055 | 29.008 | 26.8324 | 93.56% |
| Average Value | | | | | | 28.5476 | 26.7133 | 94.48% |
| Standard | | | | | | 0.683 | 0.8263 | 1.20% |
| Deviation | | | | | | | | |
| Cofficient of | | | | | | 2.39% | 3.09% | 1.27% |
| Variation (CV) | | | | | | | | |

TABLE 8
Summary of fluid handling properties

| | | | | % change |
|---|---|---|---|---|
| | Average | | | in average |
| | Free-swell | | | free-swell |
| | absorptive | Fluid retention | | absorptive |
| | capacity & | capacity & | Percent fluid | capacity |
| | standard | standard | retention & | relative to |
| | deviation | deviation | standard | non-treated |
| Sample ID | (g/100 cm²) | (g/100 cm²) | deviation | control |
| Negative Control 1 | 15.48 ± 0.88 | 14.26 ± 0.91 | 92.77% ± 3.90% | N/A |
| Non-Treated EX 2.5 mm Foam | | | | |
| (0% SAP/0% Binder) | | | | |
| Test Article 1-5IS1 | 22.72 ± 0.81 | 21.97 ± 0.75 | 97.42% ± 0.16% | +46.77% |
| (80% SAP/20% Binder) | | | | |
| Test Article 2-5IS2 | 25.61 ± 1.14 | 23.79 ± 0.65 | 94.34% ± 2.84% | +65.44% |
| (90% SAP/10% Binder) | | | | |
| Test Article 3-5IS5 | 29.12 ± 1.52 | 26.93 ± 1.37 | 93.71% ± 0.25% | +88.11% |
| (95% SAP/5% Binder) | | | | |
| Test Article 4-5IS6 | 28.55 ± 0.68 | 26.71 ± 0.83 | 94.48% ± 1.20% | +84.43% |
| (100% SAP/0% Binder) | | | | |

In summary, the use of some binding powder improves the superabsorbent polymer powder attachment/fixation to the substrate material, however, binding powder need not be provided in the bandage or dressing 10 discuss herein. Prototype material prepared without any binding powder (test article 4) exhibited a 37.6% loss of SAP powder during the fixation/shedding test. This loss was 15-28× greater than the powder loss that occurred on the other articles that contained some degree of binding powder. That being said, the concentration of binding powder may vary. All test articles that contained binding powder exhibited very low levels of powder loss (1.3-2.4%) during the fixation/shedding test. This could indicate that only a small concentration, ≤5% of binding powder, is necessary to effectively bind the SAP powder to the PU foam substrate evaluated in this study. In embodiments, other percentages of binding powder may be used, including 0%.

As the binding powder concentration increased, the stiffness or rigidity of the treated material also tends to increase, however, this characteristic may be further evaluated by measuring the flexural rigidity/bending conformability of the material via EN 1644-2, for example.

During FAC testing, concentration of binding powder appears to be negatively correlated with the absorptive capacity of the material (i.e., as the binding powder concentration increases, the absorptive capacity of the material decreases). For example, test article 2 (90% SAP/10% Binder) and test article 3 (95% SAP/5% Binder) had similar SAP concentrations, calculated as 432 gsm and 419 gsm respectively (see Table 1), thus should exhibit similar absorption capacity values. However, test article 3 consistently exhibited a >13.7% increase in absorption capacity as compared to test article 2. In embodiments, the binding powder may melt and coat the SAP powder granules to reduce absorption. The melting point for the binding powder (PEG 8000PF) is approximately 55-60° C. [4] and the thermal treatment process used to impregnate the powder into the polyurethane is performed at 100° C. for 30 seconds. Higher binding powder concentrations may result in binding powder melting and fully coating or encapsulating a larger portion of the SAP powder granules in a layer of PEG. This PEG coating may reduce the absorptive capacity of the affected SAP granules, thus resulting in an overall decrease in absorptive capacity of the treated material as the binding powder concentration increases.

With respect to the FRUC testing, the presence of superabsorbent polymer and/or binding powder appears to improve fluid retention values. All materials tested exhibited high percent fluid retention values (i.e., ≥92.7%), with superabsorbent polymer additive impregnated materials exhibiting retention values ranging from 93.7%-97.4%.

In sum, the following conclusions appear to be reasonable in view of the testing described above:
1. The use of a binding powder prevents excessive loss of the superabsorbent polymer additive powder following the powder impregnation process;
2. Increasing binding powder concentrations appears to negatively impact the stiffness, or rigidity, of the treated materials;
3. As the binding powder to superabsorbent polymer additive powder ratio increases, the absorptive capacity of the treated material decreases;
4. The presence of superabsorbent polymer additive and/or binding powder improves fluid retention values.

In embodiment, as noted above, while the use of a binding agent may improve the adhesion of the superabsorbent polymer additive to the foam layers 3-1, binding powder need not be used.

Although the present invention has been described in relation to particular embodiments thereof, many other variations and modifications and other uses will become apparent to those skilled in the art. It is preferred, therefore, that the present invention be limited not by the specific disclosure herein.

### REFERENCES

(1) BS EN 13726-1:2002, Test Methods for Primary Wound Dressings. Part 1: Aspects of Absorbency. BSI, 2002.
(2) Mennini, N., Greco, A., Bellingeri, A., De Vita, F., & Petrella, F. (2016). Quality of wound dressings: a first step in establishing shared criteria and objective procedures to evaluate their performance. Journal of wound care, 25(8), 428-437. https://doi.org/10.12968/iowc.2016.25.8.428
(3) Debashish, C., Roman, M. (2015). In Vitro Comparison of the Absorption and Retention Capabilities of Two Superabsorbent Wicking Dressings. Poster presented at the meeting of Wound, Ostomy, and Continence Nurses Society, San Antonio, TX.
(4) Carbowax™ PEG 8000 (Powder), Fisher Chemical™; ThermoFisher Scientific: Fair Lawn, NJ, Aug. 31, 2023. https://www.fishersci.com/shop/products/carbowax-pe-8000-powder-fisher-chemical-2/P156500 (Accessed Aug. 31, 2023)

What is claimed is:

1. A medical dressing comprising:
a carrier layer;
an adhesive layer provided below the carrier layer closer to a user's skin when the medical dressing is in place on the user;
an absorbent layer positioned below the adhesive layer and connected to the carrier layer via the adhesive layer,
the absorbent layer configured to absorb and retain exudate and including at least one foam layer with superabsorbent polymer additive provided on the at least one foam layer; and
a wound contact layer positioned below the absorbent layer and configured to adhere to the user's skin,
the wound contact layer including a plurality of openings through which the exudate may pass into the absorbent layer.

2. The medical dressing of claim 1, wherein the absorbent layer comprises:
a first polyurethane foam layer including at least a first layer of superabsorbent polymer additive; and
a second polyurethane foam layer including at least a second layer of superabsorbent polymer additive,
wherein the exudate is absorbed by at least one of the first layer of superabsorbent polymer additive and the second layer of superabsorbent polymer additive.

3. The medical dressing of claim 2, wherein at least one of the first layer of superabsorbent polymer additive and the second layer of superabsorbent polymer additive includes a binder powder configured to hold the superabsorbent polymer additive on the first polyurethane foam layer or the second polyurethane foam layer, respectively.

4. The medical dressing of claim 2, wherein the first layer of superabsorbent polymer additive and the second layer of superabsorbent polymer additive are provided in power form and are impregnated into the first polyurethane foam layer and the second polyurethane foam layer, respectively.

5. The medical dressing of claim 4, wherein at least one of the first layer of superabsorbent polymer additive and the second layer of superabsorbent polymer additive includes superabsorbent polymer additive provided via an aqueous solution.

6. The medical dressing of claim 4, wherein the first layer of superabsorbent polymer additive and the second layer of superabsorbent polymer additive include superabsorbent polymer additive provided via an aqueous solution.

7. The medical dressing of claim 2, wherein the first layer of superabsorbent polymer additive and the second layer of superabsorbent polymer additive are provided via an aqueous solution applied to the first polyurethane foam layer and the second polyurethane foam layer, respectively.

8. The medical dressing of claim 2, wherein the absorbent layer further comprises a third layer of superabsorbent polymer additive provided on at least one of the first polyurethane foam layer and the second polyurethane absorbent layer.

9. The medical dressing of claim 8, wherein the third layer of superabsorbent polymer additive is a powder.

10. The medical dressing of claim 8, wherein the third layer of superabsorbent polymer additive is provided via aqueous solution.

11. The medical dressing of claim 2, wherein respective edges of the first polyurethane foam layer and the second polyurethane foam layer are sealed together to create a chamber to hold the superabsorbent polymer additive in place.

12. The medical dressing of claim 11, wherein the respective edges are sealed via ultrasonic welding.

13. The medical dressing of claim 1, further comprising hydrophilic non-woven fabric bound to at least one of the first polyurethane foam layer and the second polyurethane foam layer over the superabsorbent polymer additive.

14. The medical dressing of claim 1, wherein the superabsorbent polymer additive is a powder impregnated in the at least one foam layer.

15. The medical dressing of claim 1, wherein the superabsorbent polymer additive is provided via an aqueous solution.

16. The medical dressing of claim 15, wherein the superabsorbent polymer additive remains on the at least one polyurethane foam layer after a liquid of the aqueous solution evaporates.

17. The medical dressing of claim 1, wherein the carrier layer is a polyurethane film.

18. The medical dressing of claim 1, wherein the plurality of openings formed in the wound contact layer are all the same size.

19. The medical dressing of claim 1, wherein the plurality of openings formed in the wound contact layer include a first group of openings having a large size positioned in a center of the dressing above the wound when the dressing is on the user's skin and a second group of openings having a small size positioned around a periphery of the dressing.

20. The medical dressing of claim 1, wherein a first area of the carrier layer and a second area of the wound contact layer are larger than a third area of the absorbent layer.
